(19) United States
(12) Patent Application Publication
(10) Pub. No.: US 2025/0152011 A1
(43) Pub. Date: May 15, 2025

(54) EXAMINATION DEVICE FOR OPTICAL MEDICAL EXAMINATIONS

(71) Applicant: HEINE Optotechnik GmbH & Co. KG, Gilching (DE)

(72) Inventors: Felix MICHEL, Gilching (DE); Holger Hein, Gilching (DE)

(21) Appl. No.: 18/940,982

(22) Filed: Nov. 8, 2024

(30) Foreign Application Priority Data
Nov. 10, 2023 (DE) 10 2023 131 275.8

(51) Int. Cl. A61B 5/00; H05K 7/20

(52) U.S. Cl. CPC A61B 5/0077; A61B 5/441; A61B 5/743; A61B 5/7475; H05K 7/2039

(57) ABSTRACT

An examination device for optical medical examinations, in particular a dermatoscope, including a housing, a processor and a heat sink, wherein the housing encloses an interior space, wherein the processor is located in the interior space. The heat sink includes a heat source section and a heat sink section, wherein the heat source section is located in the interior space and is in thermal contact with the processor, and wherein at least one side of the heat sink section is located outside of the interior space.

### FIELD OF THE DISCLOSURE

The disclosure relates to an examination device for optical medical examinations, in particular a dermatoscope.

### BACKGROUND

Examination devices for optical medical examinations, in particular dermatoscopes, are known that comprise a camera for examinations and a screen for displaying the recordings of the camera. Such examination devices are advantageous in comparison to purely optical examination devices as recordings can be captured and documented during the examinations.

However, it would be desirable that the examination device supports the user in the examination by displaying further information on the screen during the examination.

To be capable of providing such functionality, powerful processors comprising neural processing units, graphics processing units and/or floating-point units are needed however in the examination device. During operation, such powerful processors generate, however, a large amount of heat that must be removed from the processor and dissipated into the environment.

This is however problematic in the case of a medical examination device as known solutions for removing excess heat, such as ventilation slots, prevent the necessary thorough cleaning and disinfection or make this considerably more difficult.

### SUMMARY

It is provided an examination device for optical medical examinations, in particular a dermatoscope. The examination device comprises a housing, a head region, a grip region, a processor, a camera, a screen and a heat sink, wherein the housing encloses an interior space, and the processor is located in the interior space. The camera and the screen are located in the head region. The heat sink comprises a heat source section and a heat sink section, wherein the heat source section is located in the interior space and is in thermal contact with the processor. At least one side of the heat sink section is located outside of the interior space.

The inventors have recognised that the excess heat of the processor can be reliably and safely dissipated as a heat sink section is located outside of the interior space. Thus, the housing does not need to comprise ventilation slots or suchlike that would prevent medical cleaning and/or disinfection and thus preparation for use or make this more difficult. Moreover, dedicated regions are provided on the examination device through the head region and the grip region, and the ergonomics are improved.

Said at least one side of the heat sink section that is not located in the interior space is in particular in contact with the environment of the housing and/or is not enclosed by the housing.

The heat sink provides, for example, a heat-dissipating connection by means of thermal bridges between the heat source section and the heat sink section.

The interior space can have several subspaces.

The examination device is preferably a dedicated device, thus a device designed for this purpose. In particular, the examination device is not a telephone, smartphone, tablet or other smart device.

For example, the examination device is a handheld device, i.e. it can be held entirely by the operator during the examination.

In an embodiment, the processor comprises a neural processing unit, a graphics processing unit (GPU) and/or a floating-point unit, thereby enabling efficient image processing.

The processor can comprise a central processing unit (CPU) and/or a display interface as further units.

For example, the thermal contact between the heat source section and the processor is produced through direct physical contact or indirect physical contact using only a thermal interface material, in particular a thermal paste and/or thermal interface film.

In one aspect, the heat sink section forms a part of the exterior surface of the examination device, in particular the housing, thereby making the examination device even easier to clean.

It is also conceivable that the heat sink section is located outside of the housing.

In an embodiment, the heat sink section is located in the head region and/or in the grip region.

For example, the processor is located at the transition from the head region to the grip region to connect the processor optimally.

To increase comfort and to ensure safety with regard to prescribed contact temperatures, the examination device can comprise a patient side and an operator side, wherein the heat sink section is not located on the patient side and/or not on the operator side, and/or the examination device can have multiple lateral sides, wherein the heat sink section is located on at least one of the lateral sides.

Multiple heat sink sections can be provided. In particular, the heat sink sections can be provided on different lateral sides.

For particularly efficient heat conduction, the heat sink can be designed as a single piece or as a single-piece base with an insert, and/or the heat sink is made of metal, in particular copper, aluminium or an alloy of these.

In an embodiment, the single-piece base is made of a first metal, for example an aluminium alloy, and the insert is made of a second metal, for example copper. The insert is recessed in the base, for example in the heat source section. It is conceivable that the insert forms the heat source section.

For example, the heat source section transitions into the thermal bridge and the thermal bridge transitions into the heat sink section.

In an embodiment, the examination device comprises a frame which forms a part of the exterior surface of the housing, wherein the heat sink section is a part of the frame. In this way, the frame providing the stability of the examination device can be used simultaneously as a heat sink.

To increase the heat dissipation further, the frame and the heat sink, in particular its single-piece base, can be designed together as a single piece.

To conduct the heat into the frame in a defined manner, the heat sink can comprise a thermal bridge which connects the heat source section to the heat sink section thermally, and the thermal bridge can contact the frame at a contact point physically, in particular can transition into the frame.

The frame can extend around the contour of the examination device on the lateral sides to be particularly stable and/or comprise at least one thermal break in order to control the heat flow.

For example, the thermal break is smaller than 1 cm, in particular smaller than 0.5 cm, in the longitudinal direction in order to not impair the stability of the frame. The frame extends around the contour completely except for said one or multiple thermal breaks.

In an embodiment, a grip zone is provided on the examination device, in particular in the grip region, in particular wherein the grip zone is located between a thermal break of the frame and a contact point of the frame or is located between two thermal breaks. In this way, it is ensured that the grip zone only heats up slowly and contact temperatures are maintained safely.

It is conceivable that the grip zone is rubber-lined or covered in another way to decouple it thermally for improved haptic sensation.

For example, a gripping part can be provided in the grip zone that is designed spaced apart from the grip zone and covers the grip zone.

The contact point is located in particular on the side of the grip zone facing away from the heat source section.

For a simple construction of the housing, the housing can comprise two housing shells that are attached in particular to the frame.

To ensure the comfort and the safety of the patient, the examination device can comprise a contact zone on the patient side on the exterior surface, wherein the contact zone is thermally separated from the heat sink and/or the frame.

In an embodiment, the camera is located on the patient side and/or the camera comprises a macro lens and/or the screen is located on the operator side; and/or the examination device comprises a user interface, in particular at least one button which is located in the grip region and/or on the operator side.

The camera can be attached to the frame.

A second camera can be provided that faces forward, in particular wherein the fields of view of the two cameras do not overlap.

In an embodiment, the processor is configured to receive the recording captured by the first and/or second camera, to process the recording, in particular to execute an image analysis, and/or to control the screen in order to display the recording and, in particular, the results of the image analysis in real time. In this way, further functionalities can be provided that support the operator during the examination.

The recording is particularly a video recording. The examination can be therefore performed just like a purely optical device.

The processor can comprise at least two units, wherein one unit is configured to control the screen. Another unit can be configured to process the recording, in particular to execute an image analysis. This unit for processing the recording can be a neural processing unit and/or a floating-point unit.

The units can be located spatially separated from each other, wherein at least one unit, in particular the unit for image processing is in thermal contact with the heat source section.

The examination device can comprise an integrated energy storage device, in particular that is located in the grip region.

### BRIEF DESCRIPTION OF THE DRAWINGS

Additional features and advantages of the disclosure are found in the following description as well as the attached drawings to which reference is made. In the drawings:

FIG. 1 shows an examination device according to an embodiment of the disclosure in a perspective view from the front,

FIG. 2 shows a side view on a lateral side of the examination device according to FIG. 1.

FIG. 3 shows a perspective view of the examination device according to FIG. 1 from below, wherein the examination device is open, and

FIG. 4. shows a single-piece frame and heat sink of the examination device according to FIG. 3.

### DETAILED DESCRIPTION

Lists having a plurality of alternatives connected by “and/or”, for example “A, B and/or C” are to be understood to disclose an arbitrary combination of the alternatives, i.e. the lists are to be read as “A and/or B and/or C” or as “at least one of A, B or C”. The same holds true for listings with more than three items.

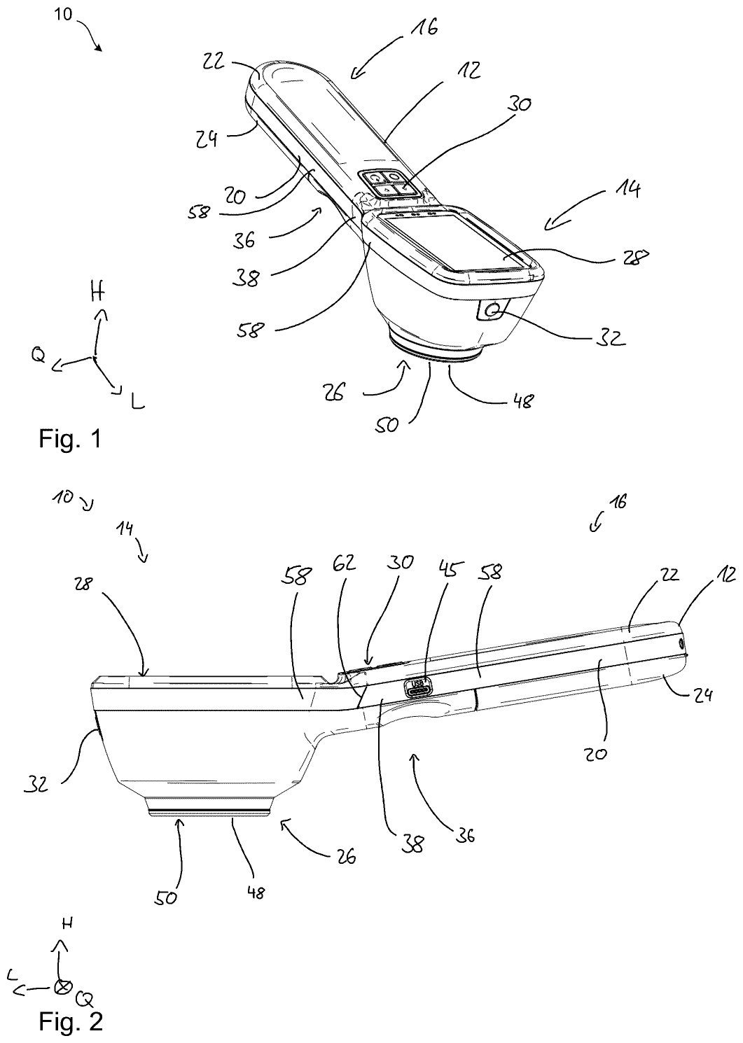

In FIG. 1, an examination device 10 for optical medical examinations is shown in perspective in a view from the top and front. Thus, the upper side of the examination device 10 can be seen primarily.

FIG. 2 shows an examination device 10 in an orthogonal side view on one of the lateral sides in the transverse direction Q.

The examination device 10 is a dermatoscope, for example, and is designed as a handheld device in the shown embodiment.

In particular, the examination device 10 is a dedicated medical examination device or dermatoscope and is designed, for example, for constant use. For example, the examination device 10 is not a telephone, smartphone, tablet or other smart device.

The examination device 10 comprises a housing 12 and has a head region 14 and a grip region 16 which adjoins the head region 14.

The examination device 10 has a longitudinal direction L, a transverse direction Q as well as a vertical direction H.

In the vertical direction H, the examination device 10 has a patient side (the lower side in in FIG. 1) and an operator side (the upper side in FIG. 1) that are opposite to each other. The remaining sides of the examination device 10 are termed lateral sides.

The grip region 16 extends in the longitudinal direction L and the head region 14 connects to this on the front side of the grip region 16.

The head region 14 is, for example, shorter than the grip region 16 in the longitudinal direction L.

The head region 14 is designed thicker in the vertical direction H, in particular more than twice as thick as the grip region 16. For example, the upper side of the grip region 16 transitions into the upper side of the head region 14, wherein the underside of the head region 14 is located offset to the underside of the grip region 16.

In the transverse direction Q, the head region 14 can be designed wider than the grip region 16.

The examination device 10 has frame 20 and two housing shells, namely an upper housing shell 22 and a lower housing shell 24.

The frame 20, the upper housing shell 22 and/or the lower housing shell 24 extend both in the head region 14 and in the grip region 16.

The housing shells 22, 24 and parts of the frame 20 are part of the housing 12, in particular they form the housing 12.

The exterior surface of the housing 12, i.e. the surfaces of the housing 12 accessible from the environment of examination device 10, are the exterior surfaces of the housing shells 22, 24 and the exterior surface of the frame 20.

For example, the upper housing shell 22 is attached to the upper side of the frame 20 and the lower housing shell 24 to the underside of the frame 20. In particular, the exterior surfaces of the housing shells 22, 24 are separated from each other through the exterior surfaces of the frame 20, as can be seen clearly in FIG. 2.

The housing 12 delimits an interior space of the examination device 10, in which further components of the examination device 10 are located. The interior space can be divided into different subspaces, in which several of the components are housed.

The examination device 10 comprises at least a camera 26, a screen 28 and a user interface 30 that is accessible from outside of the housing 12 or the environment of the examination device 10.

In the shown embodiment, the examination device comprises a first camera 26 and also a second camera 32 that are accessible from outside the housing or the environment of the examination device 10.

The first camera 26, the second camera 32 and the screen 28 are located in the head region 14.

The first camera 26 is located on the patient side of the head region 14 and has a field of view downwards through the exterior surface on the underside.

The second camera 32 is located in front in the head region 14 and has a field of view through the front lateral side of the examination device 10.

Thus, the fields of view of the first camera 26 and the second camera 32 do not overlap.

The screen 28 is located on the operator side and covers most of the operator side of the head region 14, in particular 80%.

The grip region 16 comprises a user interface 30 which is located in the form of buttons on the operator side of the grip region 16. It is also conceivable that the user interface 30 is also designed differently, for example as a touch-sensitive display and/or as part of the screen 28.

The user interface 30 is located along the grip region 16 on the end of the grip region 16 facing towards the head region 14 so that this can be operated by an operator of the examination device 10, for example, with the thumb when the operator grasps the grip region 16 with the hand.

In addition, a tapering section 36 is provided on a patient side of the grip region 16, said tapering section 36 being formed in such a way that the operator can insert, for example, the index figure into the tapering section 36 when the grip region 16 is being grasped by the hand. In this way, this results in a particularly ergonomic hand position when using the examination device 10.

A grip zone 38 on the frame 20 is provided on the lateral sides in the grip region 16, on said grip zone 38 a part of the operator's hand rests when the grip region 16 is grasped by the hand.

The grip zone 38 is, for example, in the longitudinal direction at least in the region of the tapering section 36, in the region of the user interface 30 and/or at the end of the grip region 16 facing the head region 14.

For example, two grip zones 38 are provided at lateral sides opposite to each other.

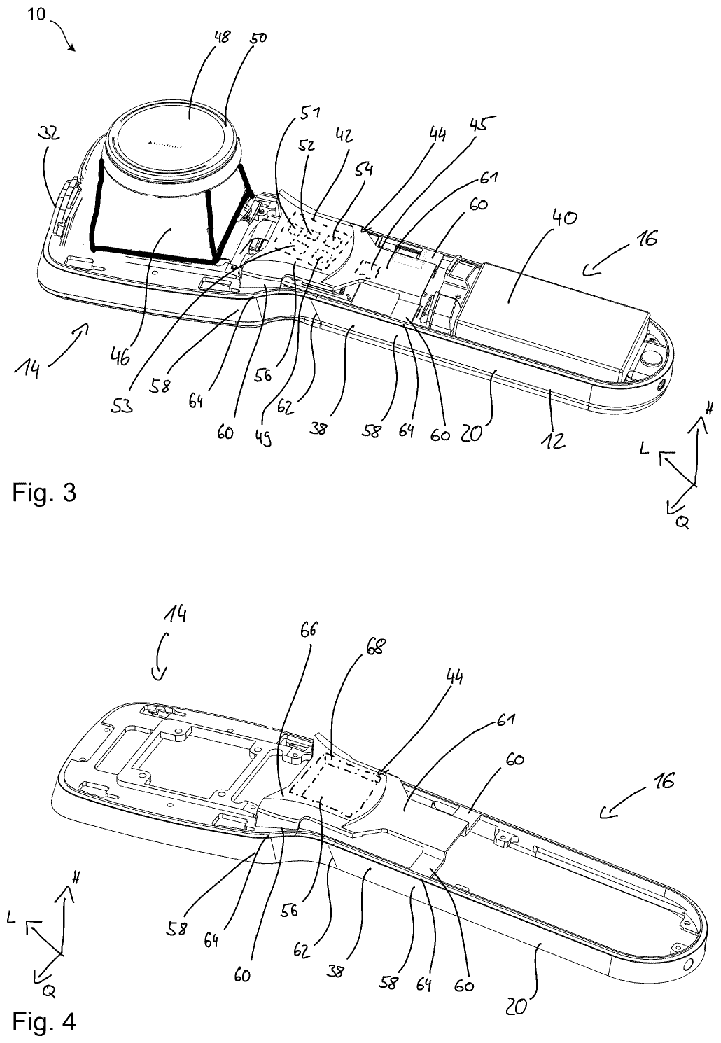

FIG. 3 shows the examination device 10 in a perspective view from below and slightly from behind, wherein the lower housing shell 24 has been removed.

In addition to the first camera 26 and the second camera 32, further components of the examination device 10 can be seen, namely an energy storage device 40, a processor 42, a heat sink 44 as well as a communication interface 45.

It can be seen in FIG. 3 that the first camera 26 comprises a macro lens 46 and thus is designed as a macro camera.

In addition, the first camera 26 has a cover glass 48 that can be placed on the patient during the examination and thus forms a contact zone 50.

The first camera 26 can be attached to the frame 20.

The integrated communication interface 45 is designed, for example, wirelessly, such as a Bluetooth module or a WLAN module. However, a wired communication interface 45, for example in the form of a USB connection (FIG. 2), is also conceivable.

The energy storage device 40 is located in the grip region 16, in particular at the rear end in the longitudinal direction L, i.e. at the end of the grip region 16 facing away from the head region 14.

In this way, the energy storage device 40 can be used simultaneously as a balancing mass within the examination device 10 to balance the weight of the head region 14 at least in part, thereby making the examination device 10 particularly precise and capable of being used by the operator with little effort.

The energy storage device 40 is a rechargeable accumulator battery.

For example, the examination device 10 is part of an examination device assembly that comprises the examination device 10 and a charging station for the examination device 10. In this case, the charging station can comprise a receiving space, into which the examination device 10 is inserted detachably in a non-destructive manner with the end of the grip region 16 having the charging interface (the rear end here) in order to execute a charging process.

Fundamentally, it is also possible that a single-use battery is provided as replaceable energy storage device 40 instead of a rechargeable energy storage device 40.

The processor 42 is located between the first camera 26 and the energy storage device 40 in the longitudinal direction L.

For example, the processor 42 is located at the transition from the head region 14 to the grip region 16. For example, the processor is placed in the longitudinal direction L in the region of the grip zone 38 or offset to the grip zone 38 to the front.

The processor 42 and the energy storage device 40 are located entirely within the housing 12, i.e. entirely within the interior space.

The processor 42 is configured to receive recordings captured by the first camera 26 and/or the second camera 32 and to control the screen 28 in such a way that this recording is displayed on the screen 28.

The recording is in particular a video recording that is reproduced in real time on the screen 28.

The processor 42 is moreover configured to not only receive and forward the recordings, but also to process the recordings, for example to execute an image analysis in real time.

During the image analysis, the processor 42 executes, for example, object and edge detection in the recordings captured. In the case of a dermatoscope, the processor 42 can recognise melanomas by means of the image analysis, measure different regions of the melanomas and/or categorise the melanomas.

As results of the image analysis, the processor 42 can mark, for example, outlines of structures, such as melanomas, in the recording and/or provide a risk assessment.

The screen 28 is controlled by the processor 42 in such a way that the results of the image analysis are displayed in real time on the screen 28 simultaneously with the recording captured, in particular superimposed with the recording.

The processor 42 can comprise at least two units, wherein on a unit is configured to process the recording, in particular execute the image analysis, and/or one unit is configured to control the screen 28.

The units can be located on a single chip or spatially separate from each other.

The processor 42 comprises as a unit, for example, a neural processing unit 52, a graphics processing unit 51 (GPU) and/or a floating-point unit 54 in order to be capable of executing the image analysis, in particular in real time.

The processor 42 can comprise as further units a central processing unit (CPU) and/or a display interface.

The heat sink 44 is provided to dissipate heat generated during operation of the processor 42. The heat sink 44 comprises a heat source section 56, a heat sink section 58 as well as a thermal bridge 60.

In the shown embodiment, the heat sink 44 has a heat source section 56, four heat sink sections 58 and four thermal bridges 60.

The thermal bridges 60 connect the heat source section 56 to the heat sink sections 58 thermally and thus provide a heat-dissipating connection.

The contact zone 50 is separated, in particular, thermally from the heat sink 44 and the frame 20.

The heat sink 44 is designed as a single piece, i.e. the heat source section 56, the heat sink sections 58 and the thermal bridges 60 are part of the same part.

The heat source section 56 thus transitions into the thermal bridges 60 that in turn transition into the corresponding heat sink sections 58.

For example, the heat sink 44 comprise a single part made of metal, in particular copper, aluminium, a copper alloy or an aluminium alloy.

In the shown embodiment, the heat sink 44 and the frame 20 are designed together as a single piece, i.e. as a single part, for example as a part made of metal. The part comprising frame 20 and heat sink 44 is shown isolated in FIG. 4.

It is evident that the frame 20 extends around the contour of the examination device 10 on the lateral sides completely. The external contour of the examination device 10 is regarded as the contour in an orthogonal view from the operator side.

The frame 20 has a thermal break 62 on each of the lateral sides in the transverse direction Q in the region of the transition from the head region 14 to the grip region 16. The thermal breaks 62 are provided, for example, in each case in the longitudinal direction L in front of the grip zone 38.

The thermal breaks 62 are each smaller than 1 cm, in particular smaller than 0.5 cm, in the longitudinal direction L. They can be designed as slots in the frame 20.

At the thermal breaks 62, the sections of the frame 20 adjoining the thermal break 62 are not in thermal contact. The thermal break 62 thus prevents heat conduction through the frame 20 at this point.

Several sections of the frame 20 form the heat sink sections 58 of the heat sink 44. The heat sink sections 58 are thus part of the frame 20.

Just like the frame 20 itself, the heat sink sections 58 are thus located outside of the interior space, at least with its exterior side. This side is therefore in contact with the environment of the housing 12 and is not enclosed by the housing 12.

The heat sink sections 58 are thus a part of the exterior surface of the examination device 10, in particular a part of the exterior surface of the housing 12.

In particular, said at least one side of the heat sink section 58 that is located outside of the interior space is in alignment with the remaining housing 12.

It is also conceivable that the heat sink sections 58 are located completely outside the housing 12, for example protruding from the housing 12.

In the shown embodiment, two heat sink sections 58 are located in the head region 14 and two heat sink sections 58 in the grip region 16. For example, in each case, a heat sink section 58 is provided in the head and grip region 14, 16 on each of the lateral sides in the transverse direction Q.

The heat sink sections 58 are thus located on the lateral sides, here the lateral sides in the transverse direction Q, and particularly not on the operator side or the patient side.

In the longitudinal direction L, one of the thermal breaks 62 is placed on each of the lateral sides in the transverse direction Q between the heat sink section 58 in the head region 14 and the heat sink section 58 in the grip region 16.

Contact points 64 of the frame 20, from which the thermal bridges 60 extend into the interior space, are situated in the heat sink sections 58.

The thermal bridges 60 contact the frame 20 at the contact points 64 thus physically.

The grip zones 38 of each of the lateral sides in the transverse direction Q are located between the corresponding thermal breaks 62 and the closest corresponding contact point 64 of the frame 20.

There is no contact point 64 between the thermal break 62 and the grip zone 38.

The grip zones 38 are situated in the longitudinal direction L from the back of the thermal break 62 and offset from the contact point 64 to the front.

The thermal bridges 60 starting from the contact points 64 in the head region 14 extend in the transverse direction Q inwards until they transition into the heat source section 56.

The thermal bridges 60 starting from the contact points 64 in the grip region 16 extend initially in the transverse direction Q inwards and subsequently in the longitudinal direction L forwards until they transition into the heat source section 56.

The section 61 of the thermal bridge 60 extending in the longitudinal direction L can be designed together.

The section 61 of the thermal bridge 60 extending in the longitudinal direction L results in making it possible to place the contact point 64 in the grip region 16 further back from the grip zone 38 although the heat source section 56 and thus the processor 42 are in the longitudinal direction L in front of the grip zone 38.

This results in a particularly long heat transfer path so that the grip zone 38 can only heat up slowly. The potential shorter heat transfer path via the contact point 64 in the head region 14 is prevented due to the thermal break 62.

It is also conceivable that the frame 20 comprises multiple thermal breaks 62 on each of the lateral sides in the transverse direction Q and the grip zone 38 is located between two of these thermal breaks 62.

The heat source section 56 is placed in the transverse direction Q centrally in the frame 20. It is situated in front of the grip zone 38 in the longitudinal direction L.

It is also conceivable in an alternative embodiment that the heat sink 44 is designed as two pieces and comprises a base 66 and an insert 68 (shown in FIG. 4 with dashed lines).

The base 66 is produced as a single piece and is made of a first metal, for example an aluminium alloy, in said base 66 the single-piece insert made of a second metal, for example copper, is inserted.

The insert 68 is located in the heat source section 56 or forms this. To this end, the base 66 can comprise a recess in the heat source section 56.

Except for the recess for the insert 68, the base 66 corresponds to the heat sink 44 previously described, in particular the base 66 together with the insert 68 corresponds completely to the heat sink 44 described previously.

If within the scope of this disclosure, it states that the heat sink 44 is designed as a single piece with the frame 20, this means for this embodiment that the base 66 is designed as a single piece with the frame 20.

As shown in FIG. 3, the processor 42, in particular the unit for image processing or the neural processing unit 52 and/or the floating-point unit 54, is provided on the heat source section 56 and is in thermal contact with this.

For example, the processor 42 (or the corresponding unit, neural processing unit 52 and/or floating-point unit 54) are attached directly to the heat source section 56, i.e. they have a direct physical contact.

It is also conceivable that there is only indirect physical contact using solely a thermal interface material between the processor 42 (or the corresponding unit, neural processing unit 52, graphics processing unit 51 and/or floating-point unit 54) and the heat source section 56. The thermal interface material can be a thermal interface film and/or a thermal paste, such as a thermal adhesive.

During the operation of the processor 42, this heats up and the heat generated is absorbed by the heat source section 56.

The heat then flows from the heat source section 56 via the thermal bridges 60 to the heat sink sections 58.

As the heat sink sections 58 are located outside the interior space and contact the environment of the housing 12, these can radiate the heat into the environment and are cooled by airflows. In this way, the processor 42 is cooled reliably.

Simultaneously, although the grip zone 38 is located very close to the processor 42, the grip zone 38 is protected from excessive heat by the choice of the placement of the contact points 64 and/or the thermal breaks 62.

It is conceivable that the grip zone 38 is rubber-lined or covered in another way to decouple it thermally for improved haptic sensation.

For example, a gripping part can be provided in the region of the grip zone 38, on said gripping part the user can hold the examination device 10. The gripping part is designed spaced apart from the grip zone 38 and covers the grip zone 38, in particular entirely. The spacing between the grip zone 38 and the gripping part is, for example, an air gap and is used for thermally insulating the gripping part. The spacing or air gap is completely closed outwards.

The gripping part can be attached to the housing 12, for example through adhesion, or be designed as part of one or both housing shells 22, 24.

Simultaneously, the frame can assume a supporting and stabilising function of the entire examination device 10 so that thus exercises a double function.

In this way, it is thus possible to provide a processor 42 that generates a lot of excess heat due to its function—for example through the neural processing unit 52 and/or the floating-point unit 54—in a holdable medical examination device, without requiring openings in the housing 12, such as ventilation openings. Any necessary hygienic preparations for use (cleaning, disinfection and/or sterilisation) of the examination device 10 are thus possible without any problems.

Moreover, increased comfort and safety of the operator or the patient is achieved through the arrangement of the grip zone 38 and the contact zone 50 as maximum contact temperatures can be maintained reliably.

1. An examination device for optical medical examinations, comprising a housing, a head region, a grip region, a processor, a camera, a screen and a heat sink, wherein the housing encloses an interior space, wherein the processor is located in the interior space,
wherein the camera and the screen are located in the head region,
wherein the heat sink comprises a heat source section and a heat sink section, wherein the heat source section is located in the interior space and is in thermal contact with the processor, and wherein at least one side of the heat sink section is located outside of the interior space.

2. The examination device according to claim 1, wherein the processor comprises at least one of a neural processing unit, graphics processing unit or floating-point unit.

3. The examination device according to claim 1, wherein the thermal contact between the heat source section and the processor is produced through direct physical contact or indirect physical contact using only one thermal interface material.

4. The examination device according to claim 1, wherein the heat sink section forms a part of an exterior surface of the examination device, or is located outside of the housing.

5. The examination device according to claim 1, wherein the heat sink section is located in at least one of the head region or the grip region.

6. The examination device according to claim 1, wherein the examination device comprises a patient side and an operator side, wherein the heat sink section is at least one of not located on the patient side and/or not located on the operator side.

7. The examination device according to claim 1, wherein the examination device has multiple lateral sides, wherein the heat sink section is located on at least one of the multiple lateral sides.

8. The examination device according to claim 1, wherein the heat sink is at least one of designed as a single piece or as a single-piece base with an insert; or made of metal.

9. The examination device according to claim 1, wherein the examination device comprises a frame which forms a part of an exterior surface of the housing, wherein the heat sink section is part of the frame.

10. The examination device according to claim 9, wherein the frame and the heat sink are designed together as a single piece.

11. The examination device according to claim 9, wherein the heat sink comprises a thermal bridge which connects the heat source section to the heat sink section thermally, and wherein the thermal bridge physically contacts the frame at a contact point.

12. The examination device according to claim 9, wherein the frame extends around a contour of the examination device on lateral sides.

13. The examination device according to claim 1, wherein a grip zone is provided on the examination device.

14. The examination device according to claim 13, wherein the grip zone is located between a thermal break of a frame of the examination device and a contact point of the frame or is located between two thermal breaks.

15. The examination device according to claim 9, wherein the housing comprises two housing shells that are attached on the frame.

16. The examination device according to claim 1, wherein the examination device comprises a contact zone on an exterior surface on a patient side, wherein the contact zone is thermally separated from at least one of the heat sink or a frame of the examination device.

17. The examination device according to claim 1, wherein the camera is located on a patient side and/or the screen is located on an operator side.

18. The examination device according to claim 1, wherein the camera comprises a macro lens.

19. The examination device according to claim 1, wherein the examination device comprises a user interface located at least one of in the grip region or on an operator side.

20. The examination device according to claim 1, wherein the processor is configured to at least one of receive a recording captured by the camera, process the recording, to control the screen in order to display the recording, or to display the recording and results of an image analysis in real time.
